(19) United States
(12) Patent Application Publication
(10) Pub. No.: US 2025/0176865 A1
(43) Pub. Date: Jun. 5, 2025

(54) ELECTRONIC RING FOR MONITORING BLOOD GLUCOSE LEVELS

(71) Applicant: ULTRAHUMAN HEALTHCARE PVT LTD, Bengaluru, Karnataka (IN)

(72) Inventors: Apoorv SHANKAR, Bangalore (IN); Yogansh NAMDEO, Bengaluru, Karnataka (IN); Anoop JAYAN, Bangalore (IN); Adhit SHET, Bangalore (IN); Vatsal SINGHAL, Bangalore (IN)

(73) Assignee: ULTRAHUMAN HEALTHCARE PVT LTD, Bengaluru, Karnataka (IN)

(21) Appl. No.: 18/851,524

(22) PCT Filed: Apr. 3, 2023
(86) PCT No.: PCT/IN2023/050322

§ 371 Date: Sep. 26, 2024

(30) Foreign Application Priority Data
Apr. 4, 2022 (IN) 202241020280

(51) Int. Cl. A61B 5/145; A61B 5/00; A61B 5/024; A61B 5/1455

(52) U.S. Cl. CPC A61B 5/14532; A61B 5/02416; A61B 5/1455; A61B 5/6826

(57) ABSTRACT

The proposed invention relates to a method of determining a blood glucose level of a user. The method comprises obtaining, by a Photoplethysmography (PPG) sensor (302), raw data related to pulsations of a user. The raw data is filtered by a Data Signal Processing (DSP) filter to obtain derived variables related to variations in blood viscosity. The raw data is further processed by a probabilistic model to obtain secondary variables related to operating conditions of a circulatory and respiratory system of the user. Readings of the PPG sensor (302) are obtained by a microcontroller (306). The readings of the PPG sensor (302) indicates intensity values of reflections of light transmitted by the PPG sensor onto a blood vessel of the user. The readings of the PPG sensor (302) are processed based on the derived variables and the secondary variables to determine a blood glucose level of the user.

### FIELD OF INVENTION

The present invention relates to monitoring of health and fitness parameters of a user by a wearable device, and specifically relates to monitoring blood glucose levels of a user by a wearable device.

### BACKGROUND

Regular health and fitness monitoring is important for accomplishing long term health and wellness goals. Blood glucose level is an important indicator of one's health especially for a diabetic and pre-diabetic user.

A user's blood glucose level fluctuates throughout the day such as in the morning, around lunch, and in the evening. During night time, when a user sleeps, the blood glucose level may drop to a dangerously low level, particularly when he is diabetic or pre-diabetic. Thus, a user may need to continuously monitor his glucose levels.

Traditionally, blood glucose monitoring is done through invasive methods including pricking the skin using a needle, such as in a lancing device, and sensing glucose level from the blood using a sensor strip. However, the traditional approaches are painful and inconvenient for day to day usage, especially when a user needs to sample one's blood several times throughout the day.

Thus, there is a need for a wearable device that determines blood glucose levels of a user throughout the day in a non-invasive and convenient manner.

### OBJECTS OF THE INVENTION

A general objective of the present invention is to offer a wearable device for measurement of blood glucose level of a user.

Another objective of the invention is to provide a cost-effective and non-invasive device for measurement of blood glucose levels.

Yet another objective of the invention is to provide an accurate method of monitoring blood glucose levels.

### SUMMARY OF THE INVENTION

The summary is provided to introduce aspects related to an electronic ring for monitoring a blood glucose levels of a user.

In one aspect, a method of determining a blood glucose level of a user may comprise obtaining raw data related to pulsations of a user by a Photoplethysmography (PPG) sensor. The raw data to obtain derived variables related to variations in blood viscosity may be filtered by a Data Signal Processing (DSP) filter. The raw data to obtain secondary variables related to operating conditions of a circulatory and respiratory system of the user may be processed by a probabilistic model. A microcontroller may obtain readings of the PPG sensor. The readings of the PPG sensor may indicate intensity values of reflections of light transmitted by the PPG sensor onto a blood vessel of the user. The microcontroller may process the readings of the PPG sensor based on the derived variables and the secondary variables to determine a blood glucose level of the user.

In one aspect, the blood glucose level may be optimized based on data obtained from an external glucose monitoring device.

In one aspect, the secondary variables may include one or more of a blood oxygen saturation level (SPO₂), heart rate variability, and blood flow dependent variables.

In one aspect, the derived variables may include parameters required for secondary and tertiary processing of the readings of the PPG sensor.

In one aspect, the readings of the PPG sensor may be obtained from the PPG sensor based on one or more triggers. The one or more triggers may include motion, time, change in viscosity of blood, and change in body temperature of the user.

In one aspect, the blood glucose level may be optimised based on data obtained from an external glucose monitoring device.

In one aspect, the blood glucose level may be optimized using baseline data. The baseline data may indicate a threshold level for physiological parameters of the user.

In one aspect, the reading of the PPG sensor is processed using one or more factors related to quality of a PPG signal received from the PPG sensor.

In one aspect, the one or more factors comprise an IR perfusion index, SPO₂, ac to de ratio, HRM PS Vpp high, and HRM PS Vpp low.

In one aspect, the electronic ring may comprise a Photoplethysmography (PPG) sensor. The PPG sensor may obtain raw data related to pulsations of a user. The raw data may be filtered using a Data Signal Processing (DSP) filter to obtain derived variables related to variations in blood viscosity. The raw data may be processed using a probabilistic model to obtain secondary variables related to operating conditions of a circulatory and respiratory system of the user. The electronic ring may comprise a microcontroller for obtaining readings of the PPG sensor. The readings of the PPG sensor may indicate intensity values of reflections of light transmitted by the PPG sensor onto a blood vessel of the user. The readings of the PPG sensor may be processed based on the derived variables and the secondary variables to determine a blood glucose level of the user.

Other aspects and advantages of the invention will become apparent from the following description, taken in conjunction with the accompanying drawings, illustrating by way of example the principles of the invention.

### BRIEF DESCRIPTION OF THE DRAWINGS

The accompanying drawings constitute a part of the description and are used to provide further understanding of the present invention. Such accompanying drawings illustrate the embodiments of the present invention which are used to describe the principles of the present invention. The embodiments are illustrated by way of example and not by way of limitation in the figures of the accompanying drawings in which like references indicate similar elements. It should be noted that references to “an” or “one” embodiment in this invention are not necessarily to the same embodiment, and they mean at least one. In the drawings:

FIG. 1 illustrates a front perspective view of an electronic ring, in accordance with an embodiment of the present invention;

FIG. 2 illustrates an exploded view of an electronic ring having a multi-layered arrangement, in accordance with an embodiment of the present invention;

FIG. 3 illustrates a side view of a middle layer/PCB used in the electronic ring, in accordance with an embodiment of the present invention;

FIG. 4(a) illustrates a top view of the electronic ring placed on a wireless charger, in accordance with an embodiment of the present invention;

FIG. 4(b) illustrates a side view of the electronic ring placed on the wireless charger, in accordance with an embodiment of the present invention;

FIG. 5 illustrates a flow chart of a method of determining a blood glucose level of a user using Machine Learning (ML) models, in accordance with an embodiment of the present invention; and

FIG. 6 illustrates a normal probability plot depicting final glucose levels measured using different sample percentile, in accordance with an embodiment of the present invention.

### DETAILED DESCRIPTION OF THE INVENTION

The detailed description set forth below in connection with the appended drawings is intended as a description of various embodiments of the present invention and is not intended to represent the only embodiments in which the present invention may be practiced. Each embodiment described in this disclosure is provided merely as an example or illustration of the present invention, and should not necessarily be construed as preferred or advantageous over other embodiments. The detailed description includes specific details for the purpose of providing a thorough understanding of the present invention. However, it will be apparent to those skilled in the art that the present invention may be practiced without these specific details.

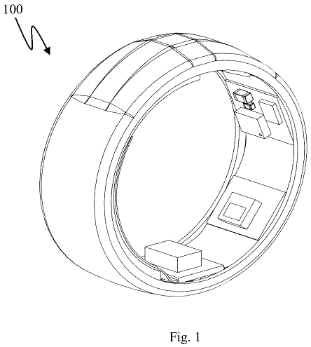

The proposed invention relates an electronic ring for monitoring blood glucose levels of users. The electronic ring may be worn over a finger. FIG. 1 illustrates a front perspective view of an electronic ring (100), in accordance with an embodiment of the present invention. The electronic ring (100) may be developed in different sizes to fit over different fingers of different users. The electronic ring (100) may include different sensors for monitoring the health and fitness parameters of users. Types, placement, and working of the sensors used in the electronic ring (100) is described henceforth.

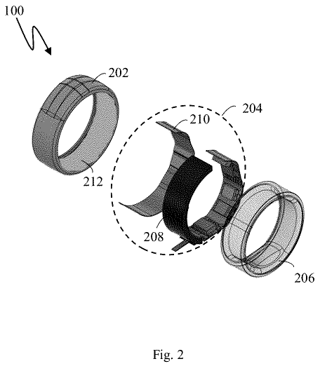

FIG. 2 illustrates an exploded view of the electronic ring (100) having multi-layered arrangement, in accordance with an embodiment of the present invention. The electronic ring (100) may comprise an outer layer (202), a middle layer (204), and an inner layer (206). The outer layer (202) may be made of a rigid, antirust or thermally conductive material, or a material having all such properties. The middle layer (204) may be positioned between the outer layer (202) and the inner layer (206). The middle layer (204) may include a Printed Circuit Board (PCB) (208) and a battery (210). The PCB (208) may be flexible, semi-flexible, or rigid. The PCB (208) may house one or more sensors to capture a plurality of health and fitness parameters of a user.

The inner layer (206) of the electronic ring (100) positioned below the PCB (208) may come in contact of the user's finger once the user wears the electronic ring (100). The inner layer (206) may be made of a semi-transparent, translucent, or completely transparent, water-resistant material, such as glass, plastic, resin, or silicone. The inner layer (206) may be transparent to a wide range of wavelengths in the electromagnetic spectrum.

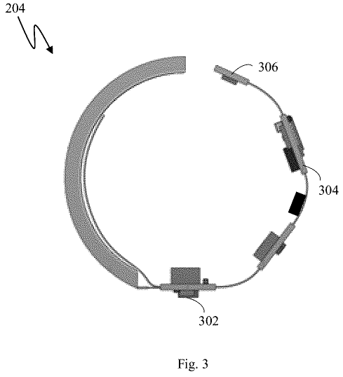

FIG. 3 illustrates a side view of the middle layer i.e. the PCB (204), in accordance with an embodiment of the present invention. As illustrated in FIG. 3, the PCB (204) may include a PhotoPlethysmoGraphy (PPG) sensor (302). The PPG sensor (302) may measure volumetric variations of blood circulation of the user. The PPG sensor (302) may comprise one or more light sources and photodetectors. The one or more light sources may focus lights signals of different wavelengths over tissues, blood vessels, and arteries of the user. The lights signals of different wavelengths may fall in visible or near visible spectrum, such as red light, green light, and infrared. The light signals transmitted through blood of the user or reflected from the tissues, blood vessels, and arteries of the user may be measured through the one or more photodetectors of the PPG sensor (302). The one or more photodetectors of the PPG sensor (302) may be placed at multiple locations throughout the inner layer (206) to capture the light signals reflected or transmitted from blood vessels or tissues of the user. Focussing of the light signals and capturing of reflection and transmission of the light signals, by the PPG sensor (302), may be done through the inner layer (206). The inner layer (206) may be made transparent to allow the PPG sensor (302) to obtain precise readings, from the finger of the user.

In one embodiment, the PPG sensor (302) may transmit light/signal in a radio wave range as different wavelengths of light may be absorbed and transmitted differently by blood glucose molecules. For example, the PPG sensor (302) may emit light in Infrared (IR) and/or red spectrum onto the skin of the user. Readings of the PPG sensor (302) from multiple such wavelengths may be processed to predict glucose level with higher accuracy.

For predicting the glucose level, a crest factor of a PPG signal for both light wavelengths may be calculated. The crest factor may be a measure of the peak-to-average ratio of the PPG signal. An IR DC sample and a RED DC sample of the user may also be measured for calculating the baseline for all calculations.

Various factors, such as an IR perfusion index, SPO₂, ac to dc ratio, HRM PS Vpp high, and HRM PS Vpp low may be measures. The factors may indicate quality of the PPG signal received by the PPG sensor (302). For example, the HRM PS Vpp high and the low refer to the maximum and minimum values of the voltage peak-to-peak measurement over a given period of time, the HRM PS Vpp high and the low may be used to assess the strength and consistency of the pulsatile waveform signal. A higher HRM PS Vpp high value indicates a stronger and more consistent pulsatile waveform signal, while a lower HRM PS Vpp low value indicates a weaker and less consistent signal. The factors related to quality of the PPG signal may contribute to the final correction applied to a final measured PPG signal which is finally used to calculate a glucose value prediction. Further, the glucose value prediction may be processed through a combination of Statistical Regression Analysis and the Supervised Machine Learning Techniques trained on data acquired from the CGM for each user for calculating a final glucose value. Thus, the factors related to quality of the PPG signal measured along with the PPG signal may also contribute to determine the final glucose value.

Blood glucose levels in human body is correlated to blood viscosity and blood flow rate. Higher blood viscosity may indicate of diabetic condition of the user. For an example, a change in mean value of blood glucose from 100 mg/dL to 400 mg/dL may cause an increase in blood viscosity by 25%, a decrease in blood flow rate by 20%, and an increase in Blood Pressure (BP) by 25% for physiological compensation. Therefore, the PPG sensor (302) may be utilised to monitor of blood glucose levels of the user by measuring blood flow variations.

The PCB (204) may further include a microcontroller (304). The PPG sensor (302) mounted on the flexible PCB (204) may be connected to the microcontroller (304), to provide raw data captured by the PPG sensor (302) for determining values of glucose levels.

The PCB (204) may be connected with an external user device through a wireless module (306) for communicating with an external user device. The wireless module (306) may work on one or more of Bluetooth and Near Field Communication (NFC). The wireless module (306) may be mounted on the PCB (204) to wirelessly communicate the values of glucose levels to the external user device, such as a smartphone or a laptop. The external user device may act as a notification means for the user to access readings of the plurality of health and fitness parameters in a visual or audible format. In another implementation, the PCB (20) may be configured to connect with the external user device through a cloud based platform via a network(s).

The battery (210) may be used to power the PPG sensor (302), the micro-controller (304), and the wireless module (306) present in the electronic ring (100).

In another implementation, the PCB (204) may be configured to connect with the external user device through a cloud based platform via a network(s).

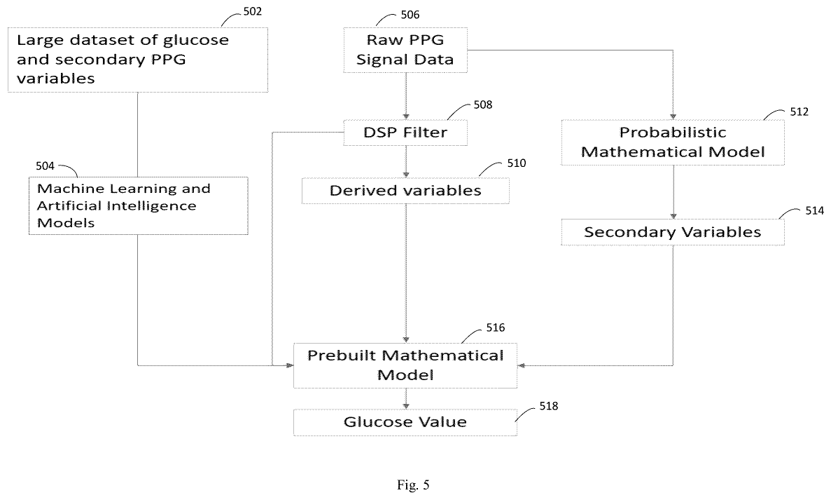

The PPG sensor (302) may transmit the raw data in response to a triggering event. The triggering event may include internal triggers and external triggers. The internal triggers may be time based triggers generated at pre-defined time intervals for initiation of PPG signal recordings. The PPG signal recorded at pre-defined time interval may provide a baseline value for the blood glucose of the user. External triggers may include triggers based on motion, time of day, change in body temperature, and change in viscosity of blood. The external triggers may be caused by factors such as physical activity of the user and food consumption. The microcontroller (304) may store the baseline data collected for physiological parameters of the user. The physiological parameters may include blood viscosity, blood pressure, and body temperature of the user. The external triggers may be detected by monitoring the baseline data for a deviation in threshold levels of the physiological parameters. The microcontroller (304) may also store the raw data in its own memory or a separate memory element mounted on the PCB (204). The microcontroller (304) may process the raw data to determine a value of glucose level of the user. The process of determining of the value of glucose level is described later with reference to FIG. 5.

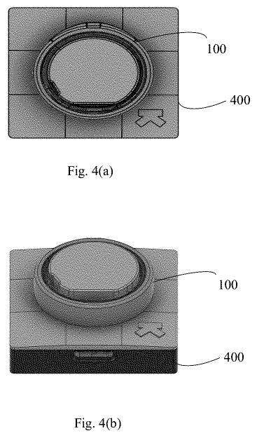

FIGS. 4(a) and 4(b) illustrate top view and side view respectively of the electronic ring (100) placed on a wireless charger (400), in accordance with an embodiment of the present invention. The electronic ring (100) may be wirelessly chargeable using the wireless charger (400). For wireless charging, the electronic ring (100) may comprise a wireless charging coil. The wireless charging coil may be positioned above or below the PCB (204). Electromagnetic field generated by a coil present in the wireless charger (400) may get coupled with the wireless charging coil of the electronic ring (100) when the electronic ring (100) is present above the wireless charger (400). Through coupling of the electromagnetic field, power may be received and stored in the battery (308). The wireless charger (400) may itself include a battery of capacity sufficient to charge the battery (210) of the electronic ring (100) a few times.

FIG. 5 illustrates a flow chart of a method of determining a blood glucose level of a user using Machine Learning (ML) models, in accordance with an embodiment of the present invention. The data model may be pre-trained prior to usage. For training of the ML models, a large dataset of reflected light signals captured by the PPG sensor (302) from different users may be collected, at step 502. The dataset may be utilised to train ML models, at step 504. The ML models may include, but not limited to, supervised machine learning models such as linear classifiers, Support Vector Machines (SVMs), decision trees, k-nearest neighbour, random forest, and Bayesian classification. The dataset may be used for optimising the blood glucose level based on the PPG readings obtained from the PPG sensor (302).

Raw PPG signal data related to pulsations of the user may be obtained, at step 506. The raw PPG signal data may be captured by emitting lights of different wavelengths, such as red light, green light, and infrared light onto the user. The light reflected by blood cells of the user may be obtained to form the raw PPG signal data. The raw PPG signal data may be filtered using Data Signal Processing (DSP) filter, such as a finite impulse response filter and an infinite impulse response filter, at step 508. Derived variables may be obtained through filtering of the raw PPG signal data, at step 510. Derived variables may include parameters required for secondary and tertiary processing of PPG readings to obtain blood viscosity variations.

For enhancing accuracy of determination of the blood glucose level, the raw PPG signal data may be processed using a probabilistic model, at step 512. Secondary variables may be obtained by processing of the raw PPG signal data, at step 514. The secondary variables may indicate operating conditions of a circulatory and respiratory system of the user, such as an inter pulse interval. The inter pulse interval, calculated using the heart rate algorithm, may be required in calculating blood flow rate. The secondary variables may include variables such as blood oxygen saturation level (SPO₂), heart rate variability, and blood flow dependent variables.

Further, a prebuilt mathematical model may be executed on the reading of the PPG sensor (302) based on the derived variables and the secondary variables, at step 516. In an implementation, the prebuilt mathematical model may be made by combining the ML models pre-trained using dataset of reflected light signals captured by the PPG sensor (302) from different users. The blood glucose level may be obtained by execution of the prebuilt mathematical model on the reading of the PPG sensor (302), at step 518.

A mathematical model was developed by collecting CGM data from each user and training them against the individual reflection readings of each LED from the PPG sensor (302) obtained from the electronic ring (100).

The mathematical model was developed with a combination of Regression Analysis and Supervised Learning. While regression analysis was used to model the relationship between glucose levels and the reflections obtained from each LED, various signal quality parameters and identify the weight functions for each of these factors to describe the relationship between the variables. The supervised machine learning technique was used to train the mathematical model using the labelled and weighted factors data and the PPG signal values with the corresponding glucose level obtained from the CGM. The mathematical model then learns to associate the PPG signal and the signal quality parameters with the glucose level and use this association to predict the glucose level of the user in the future.

The mathematical model received the data at 1 minute intervals from both the CGM and the individual LED values of the PPG sensor including the signal quality parameters from the sensor. The signal is pre-processed to remove any noise and artifacts, normalizing, and scaling the data, and aligning the data in time to the CGM data values. The weighted signal quality parameters and the 3 LED signals are then assigned a correlation parameter (between +1 and −1) to the Glucose Values. A neural network was trained using the training data. The parameters of the model were optimized to be able to accurately predict glucose levels based on the extracted features. The mathematical model was then trained using some part of the data, validated by the same and a different set of data and tested by another independent set of data. The hyper-parameters of the model were tuned using a grid search to further to optimize the performance of the model.

This model was deployed on the wearable to a large number of users simultaneously using the wearable and the CGM. The algorithm was trained over a period of 6 months using these readings to fine tune values. The PPG signal quality parameters for every individual user including the skin colour parameters were used to train the machine learning model to experimentally determine the factors to be included in the glucose value prediction.

Effects of the factor related to the PPG signal of determination of the final glucose value were observed and results were captured in Table 1 through Table 4 as provided below.

TABLE 1
Regression Statistics

| | |
|---|---|
| Multiple R | 1 |
| R Square | 1 |
| Adjusted R Square | 65535 |
| Standard Error | 0 |
| Observations | 6 |

TABLE 2

| ANOVA | df | SS | MS |
|---|---|---|---|
| Regression | 9 | 1527.333 | 169.7037 |
| Residual | 0 | 0 | 65535 |
| Total | 9 | 1527.333 | |

TABLE 3

| | | Standard | t | Lower | Upper | Lower | Upper |
|---|---|---|---|---|---|---|---|
| | Coefficients | Error | Stat | 95% | 95% | 95.0% | 95.0% |
| Intercept | 65.9597315 | 0 | 65535 | 65.9597315 | 65.95973 | 65.95973 | 65.95973 |
| spo2_crest_factor | 0 | 0 | 65535 | 0 | 0 | 0 | 0 |
| spo2_ir_dc_sample | 0.010815361 | 0 | 65535 | 0.010815361 | 0.010815 | 0.010815 | 0.010815 |
| spo2_red_dc_sample | −0.005690839 | 0 | 65535 | −0.005690839 | −0.00569 | −0.00569 | −0.00569 |
| spo2_dc_to_ac_ratio | −0.196960274 | 0 | 65535 | −0.196960274 | −0.19696 | −0.19696 | −0.19696 |
| spo2_ir_perfusion_index | 0 | 0 | 65535 | 0 | 0 | 0 | 0 |
| hrm_ps_vpp_high | 0 | 0 | 65535 | 0 | 0 | 0 | 0 |
| hrm_ps_vpp_low | 0.034366172 | 0 | 65535 | 0.034366172 | 0.034366 | 0.034366 | 0.034366 |
| hrm_crest_factor | 0 | 0 | 65535 | 0 | 0 | 0 | 0 |
| hrm_perfusion_index | −0.321983573 | 0 | 65535 | −0.321983573 | −0.32198 | −0.32198 | −0.32198 |

TABLE 4
PROBABILITY OUTPUT

| Percentile | glucose cgm |
|---|---|
| 8.333333333 | 100 |
| 25 | 106 |
| 41.66666667 | 109 |
| 58.33333333 | 134 |
| 75 | 136 |
| 91.66666667 | 139 |

This model is trained on these same parameters for a period of 15 days for every new user to further train the model for the best output.

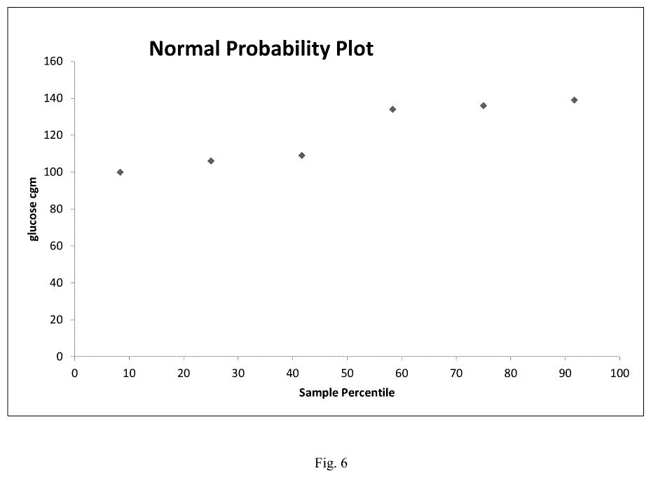

FIG. 6 illustrates a normal probability plot depicting final glucose levels measured using different sample percentile, in accordance with an embodiment of the present invention. The normal probability plot shows an effect of the factor related to the PPG signal of determination of the final glucose value.

In one implementation, the prebuilt mathematical model may be developed/trained on server prior to usage. In another implementation, the electronic ring (100) may be calibrated to a single user for accurate monitoring of the blood glucose level. The calibration of the electronic ring (100) may be performed using an external glucose monitoring device. The external glucose monitoring device may be a Continuous Glucose Monitoring (CGM) device worn by the user. The CGM may measure an amount of glucose in the fluid inside user's body. The CGM may be connected to an external user device. Blood glucose level determined by the electronic ring (100) based on the PPG sensor (302) and the data from the CGM may be transmitted to the external user device. The external user device may process and calibrate the blood glucose level from the prebuilt mathematical model to get more accurate results.

The prebuilt mathematical model may initially collect user data such as location of the user and gender. The prebuilt mathematical model may be trained on a data from a large number of users classified based on skin colour, gender, skin hydration levels, location of the user, climatic variations of the user, and dietary variations of the user. Dietary variations of the user may be collected from food logging in software application in the external device. Correction parameters for the PPG sensor values may then be calculated based on the user data. Such calibration of the PPG reading may happen during the first few readings taken by the user and may happen after every few hours to avoid reporting of incorrect readings. In an implementation, calibration of the PPG readings may be performed for a specified time period, such as fifteen days after wearing the electronic ring (100). The calibration may be periodically repeated for the specified time period in a per-defined interval, such as every 6 months to verify and improve prediction accuracy of the blood glucose level.

The prebuilt mathematical model may calibrate itself by taking data from the CGM and the mobile application to adjust its correction factors continuously to adjust its prediction values to match those values measured by the CGM. This continuous learning and adjustment of the correction factors applied to the secondary and derived variables lead to accurate prediction of the blood glucose level.

In another implementation, confidence on the PPG readings may be calculated using the auto-correlation function to study variations in blood viscosity based on hydration level of the user. The value of the auto-correlation function may vary between +1 and −1. The variation in the auto-correlation function value may be closely studied to determine variations in the body hydration. The values obtained by the PPG sensor (302) in a well hydrated body has good confidence levels and hence an autocorrelation function value which is closer to +1. A dehydrated body may have lower confidence values and hence an autocorrelation function value approaching 0.

The electronic ring (100) may be worn by a user at all times so that blood glucose levels are continuously tracked and reported to him. The electronic ring (100) provides an accurate means for tracking and logging blood glucose levels of the user in real time. With the data obtained from the electronic ring (100), the user may be able to track changes in his lifestyle, activities, and habits, thereby enabling the user to make conscious data driven lifestyle changes to improve one's health and fitness.

In the above detailed description, reference is made to the accompanying drawings that form a part thereof, and illustrate the best mode presently contemplated for carrying out the invention. However, such description should not be considered as any limitation of scope of the present unit. The structure thus conceived in the present description is susceptible of numerous modifications and variations, all the details may furthermore be replaced with elements having technical equivalence.

1. A method of determining a blood glucose level of a user, the method comprising:
obtaining, by a Photoplethysmography (PPG) sensor (302), raw data related to pulsations of a user;
filtering, by a Data Signal Processing (DSP) filter, the raw data to obtain derived variables related to variations in blood viscosity;
processing, by a probabilistic model, the raw data to obtain secondary variables related to operating conditions of a circulatory and respiratory system of the user;
obtaining, by a microcontroller (306), readings of the PPG sensor (302), wherein the readings of the PPG sensor (302) indicates intensity values of reflections of light transmitted by the PPG sensor (302) onto a blood vessel of the user; and
processing, by the microcontroller (306), the readings of the PPG sensor (302) based on the derived variables and the secondary variables to determine a blood glucose level of the user.

2. The method as claimed in claim 1, wherein the readings of the PPG sensor (302) is obtained from the PPG sensor (302) based on one or more triggers, and wherein the one or more triggers include motion, time, change in viscosity of blood, and change in body temperature of the user.

3. The method as claimed in claim 1, further comprising optimising the blood glucose level based on data obtained from an external glucose monitoring device.

4. The method as claimed in claim 3, wherein the blood glucose level is optimized using baseline data, and wherein the baseline data indicates a threshold level for physiological parameters of the user.

5. The method as claimed in claim 1, wherein the secondary variables includes one or more of a blood oxygen saturation level (SPO₂), heart rate variability, and blood flow dependent variables.

6. The method as claimed in claim 1, wherein the derived variables include parameters required for secondary and tertiary processing of the readings of the PPG sensor (302).

7. The method as claimed in claim 1, wherein the reading of the PPG sensor (302) is processed using one or more factors related to quality of a PPG signal received from the PPG sensor (302).

8. The method as claimed in claim 7, wherein the one or more factors comprise an IR perfusion index, SPO₂, ac to dc ratio, HRM PS Vpp high, and HRM PS Vpp low.

9. An electronic ring (100) for monitoring a blood glucose level of a user, the electronic ring (100) comprising:
a Photoplethysmography (PPG) sensor (302) for obtaining raw data related to pulsations of a user, wherein the raw data is filtered using a Data Signal Processing (DSP) filter to obtain derived variables related to variations in blood viscosity, and wherein the raw data is processed using a probabilistic model to obtain secondary variables related to operating conditions of a circulatory and respiratory system of the user; and
a microcontroller (306) for:
obtaining readings of the PPG sensor (302), wherein the readings of the PPG sensor (302) indicates intensity values of reflections of light transmitted by the PPG sensor onto a blood vessel of the user; and
processing the readings of the PPG sensor (302) based on the derived variables and the secondary variables to determine a blood glucose level of the user.

10. The electronic ring (100) as claimed in claim 9, wherein the blood glucose level is optimized based on data obtained from an external glucose monitoring device.

11. The electronic ring (100) as claimed in claim 9, wherein the secondary variables includes one or more of a blood oxygen saturation level (SPO₂), heart rate variability, and blood flow dependent variables.

12. The electronic ring (100) as claimed in claim 9, wherein the derived variables include parameters required for secondary and tertiary processing of the readings of the PPG sensor (302).

13. The electronic ring (100) as claimed in claim 9, wherein the reading of the PPG sensor (302) is processed using one or more factors related to quality of a PPG signal received from the PPG sensor (302).

14. The electronic ring (100) as claimed in claim 13, wherein the one or more factors comprise an IR perfusion index, SPO₂, ac to dc ratio, HRM PS Vpp high, and HRM PS Vpp low.
